(19) United States
(12) Patent Application Publication
(10) Pub. No.: US 2009/0182367 A1
(43) Pub. Date: Jul. 16, 2009

(54) Adjustable Width Trocar

(76) Inventor: Dyson W. Hickingbotham, Stouchsberg, PA (US)

Correspondence Address: ALCON, IP LEGAL, TB4-8, 6201 SOUTH FREEWAY, FORT WORTH, TX, 76134, US

(21) Appl. No.: 12/024,330

(22) Filed: Feb. 1, 2008

Related U.S. Application Data
(60) Provisional application No. 61/020,235, filed on Jan. 10, 2008.

(51) Int. Cl. A61B 17/34

(52) U.S. Cl. 606/185

(57) ABSTRACT

A trocar having a blade split into two portions, a fixed portion and a movable portion. The movable portion is attached to the fixed portion at a pivot point that allows the movable blade portion to extend beyond the width of the fixed portion or be retracted within the width of the fixed portion. The cannula may be slidably received on the blade and provide the means to retract the movable portion of the blade to within the width of the fixed portion of the blade.

This application claims the benefit of U.S. Provisional Application No. 61/020,235 filed Jan. 10, 2008.

### BACKGROUND OF THE INVENTION

Microsurgical instruments typically are used by surgeons for any manipulations or removal of tissue from delicate and restricted spaces in the human body, particularly in surgery on the eye, and more particularly in procedures for manipulations or removal of the vitreous body, blood, scar tissue, or the crystalline lens. Such instruments may be hand-held, but often include a control console and a surgical handpiece with which the surgeon dissects, manipulates and/or removes the tissue. The handpiece has a surgical tool such as a vitreous cutter probe or an ultrasonic fragmentor for cutting or fragmenting the tissue and is connected to the control console by a long air pressure (pneumatic) line or power cable and by long conduits, cable, optical cable or flexible tubes for supplying an infusion fluid to the surgical site and for withdrawing or aspirating fluid and cut/fragmented tissue from the site. The cutting, infusion and aspiration functions of the handpiece are controlled by the remote control console that not only provides power for the surgical handpiece(s) (e.g., a reciprocating or rotating cutting blade or an ultrasonically vibrated needle), but also controls the flow of infusion fluid and provides a source of reduced pressure (relative to atmosphere) for the aspiration of fluid and cut/fragmented tissue. The functions of the console are controlled manually by the surgeon, usually by means of a foot-operated switch or proportional control.

During posterior segment surgery the surgeon typically uses several instruments. This requires that these instruments be inserted into and removed out of the incision. This repeated removal and insertion can cause trauma to the eye at the incision site. To address this concern, cannulae were developed by the mid-1980s. These devices consist of a narrow tube with an attached hub. An incision is made, and the tube is inserted into the incision up to the hub, which acts as a stop, preventing the tube from entering the eye completely. Surgical instruments can be inserted into the eye through the tube, and the tube protects the incision from repeated contact by the instruments. In addition, the surgeon can use the instrument, by manipulating the instrument when the instrument is inserted into the eye through the tube, to help position the eye during surgery.

Prior art cannulae are necessarily small in diameter, but are generally available in three diameters, 20 gauge, 23 gauge and 25 gauge. The size used by the surgeon depends upon several factors, including the surgical technique and the procedure being performed. Prior to insertion of the cannula, an appropriately sized incision must first be made with a trocar. Prior art cannulae usually are sold in conjunction with the appropriate trocar. However, some surgeons prefer that the incision be slightly larger than the incision provided by the trocar. Prior to the present invention, widening the initial trocar incision required the use of a second knife in a second step. This requires additional time to make the widening incision as well as the additional expense of purchasing a second knife.

Accordingly, a need continues to exist for a trocar having a variable blade width that allows the surgeon to choose the size of the incision to be made.

### BRIEF SUMMARY OF THE INVENTION

The present invention improves upon prior art by providing a trocar having a blade split into two portions, a fixed portion and a movable portion. The movable portion is attached to the fixed portion at a pivot point that allows the movable blade portion to extend beyond the width of the fixed portion or be retracted within the width of the fixed portion. The cannula may be slidably received on the blade and provide the means to retract the movable portion of the blade to within the width of the fixed portion of the blade.

Accordingly, an objective of the present invention is to provide an ophthalmic trocar.

Another objective of the present invention is to provide an ophthalmic trocar having a blade with a fixed portion and a movable portion.

A further objective of the present invention is to provide an ophthalmic trocar having a blade with a movable portion that causes the blade to have a variable width.

Other objectives, features and advantages of the present invention will become apparent with reference to the drawings, and the following description of the drawings and claims.

### BRIEF DESCRIPTION OF THE DRAWINGS

FIG. 1 is a perspective view of distal end of the trocar of the present invention with the blade in the relaxed or unconfined state.

FIG. 2 is a perspective view of distal end of the trocar of the present invention with the blade being withdrawn from the cannula and the blade is compressed to a confined state.

FIG. 3 is an enlarged perspective view of distal end of the trocar of the present invention with the blade in the relaxed or unconfined state taken at circle 3 in FIG. 1.

FIG. 4 is an enlarged top plan view of distal end of the trocar of the present invention with the blade in the relaxed or unconfined state.

### DETAILED DESCRIPTION OF THE PREFERRED EMBODIMENTS

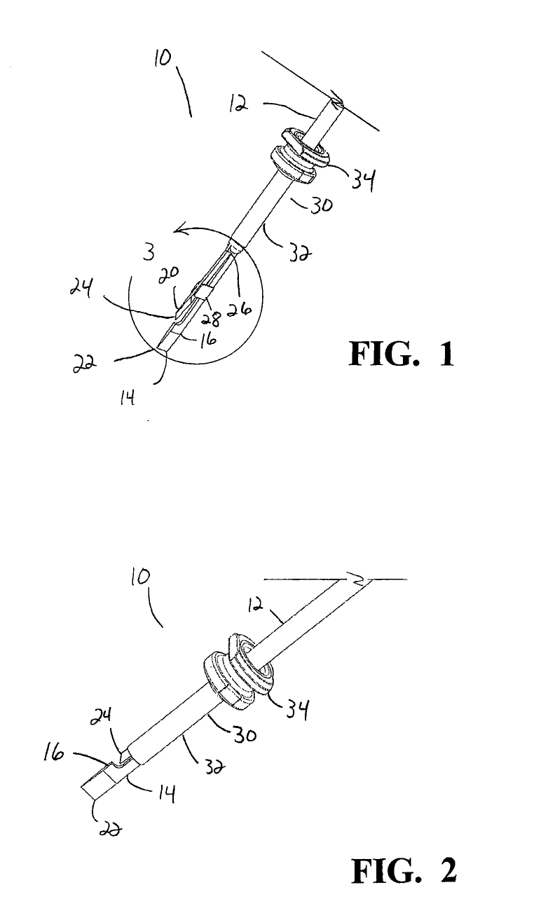

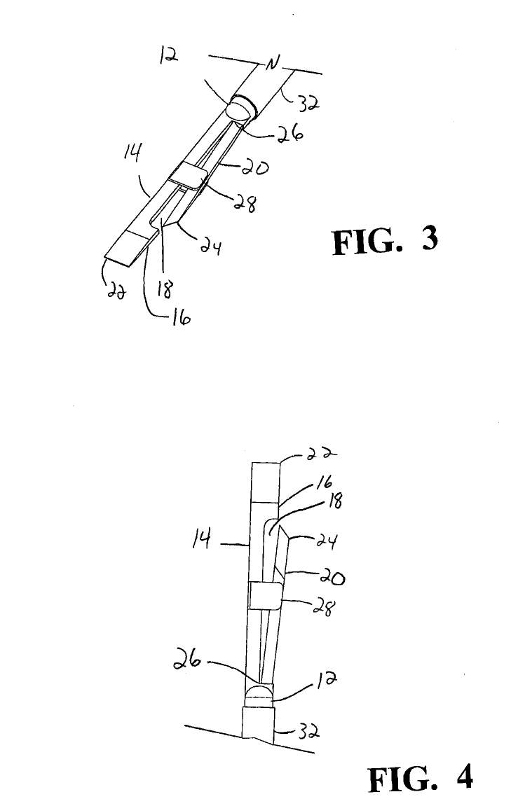

As best seen in FIGS. 1, 3 and 4, trocar 10 of the present invention generally consists of shaft 12 having blade 14. Blade 14 has two portions, fixed portion 16 having recess 18 and movable or extendable portion 20. Fixed portion 16 has sharpened edge 22 and movable portion 20 has sharpened edge 24. Both edges 22 and 24 are suitable for cutting or penetrating tissue such as ocular tissue. Fixed portion 16 and movable portion 20 are attached at spring hinge 26 that forces movable portion 20 out of recess 18 and away from fixed portion 16 in the relaxed or uncompressed state. As best seen in FIG. 2, when spring hinge 26 is compressed, movable portion 20 is forced within recess 18 of fixed portion 16. Blade guides 28 assist in keeping movement of movable portion 20 within the plane of blade 14.

In use, cannula 30 having tube 32 and hub 34 is slidably received on shaft 12 of trocar 10. Suitable cannulac 30 are well known in the art and are commercially available from Alcon Laboratories, Inc., Fort Worth, Tex. As best seen in FIGS. 1, 3 and 4, when cannula 30 is retracted proximally along shaft 12 away from blade 14, spring hinge 26 forced movable portion 20 of blade 14 out of recess 18 in fixed portion 16. As best seen in FIG. 2, when cannula 30 is extended distally along shaft 12, tube 32 forces movable portion 20 of blade 14 to retract within recess 18 of fixed portion 16 of blade 14 by bending spring hinge 26. One skilled in the art will recognize that by varying the location of tube 32 on shaft 12, the position of movable portion 20 of blade 14 may also be varied, resulting in the overall width of blade 14 to vary accordingly. For example, when movable portion 20 is fully retracted within recess 18 of fixed portion 16, the overall width of blade 14 may be suitable for making an incision appropriate for cannula 30 forming a part of a 25 gauge instrumentation system. When movable portion 20 is fully extended beyond recess 18 of fixed portion 16, the overall width of blade 14 may be suitable for making an incision appropriate for cannula 30 forming a part of a 20 gauge instrumentation system.

While certain embodiments of the present invention have been described above, these descriptions are given for purposes of illustration and explanation. Variations, changes, modifications and departures from the systems and methods disclosed above may be adopted without departure from the scope or spirit of the present invention.

I claim:

1. A trocar, comprising:
a) a shaft; and
b) a blade connected to the shaft, the blade having a fixed portion and a movable portion, the movable portion attached to the fixed portion by a spring hinge.

2. The trocar of claim 1 wherein the fixed portion further comprises a recess into which the movable portion fits upon compression of the spring hinge.

3. The trocar of claim 1 further comprising a blade guide for guiding the movement of the movable portion relative to the fixed portion.

4. A trocar, comprising:
a) a shaft;
b) a blade connected to the shaft, the blade having a fixed portion and a movable portion, the movable portion attached to the fixed portion by a spring hinge;
c) a recess into which the movable portion fits upon compression of the spring hinge; and
d) a blade guide for guiding the movement of the movable portion relative to the fixed portion.
